(19) United States
(12) Patent Application Publication
(10) Pub. No.: US 2021/0308094 A1
(43) Pub. Date: Oct. 7, 2021

(54) CESSATION OF ANTI-DEPRESSANT MEDICATION

(71) Applicant: Resurgent Biosciences, Inc., Minneapolis, MN (US)

(72) Inventors: Eric Greenbaum, New York, NY (US); Kyle Kingley, Minneapolis, MN (US)

(73) Assignee: Resurgent Biosciences, Inc., Minneapolis, MN (US)

(21) Appl. No.: 17/351,590

(22) Filed: Jun. 18, 2021

Related U.S. Application Data
(63) Continuation of application No. 15/436,480, filed on Feb. 17, 2017.
(60) Provisional application No. 62/296,410, filed on Feb. 17, 2016.

(51) Int. Cl. A61K 31/352; A61K 36/185; A61K 31/047; A61P 25/30

(52) U.S. Cl. CPC A61K 31/352; A61P 25/30; A61K 31/047; A61K 36/185

(57) ABSTRACT

A medicament including cannabidiol (CBD) and delta-9-tetrahydrocannabinol (THC) for mitigating and/or alleviating one or more symptoms associated with cessation of antidepressant medication are provided. For example, the present disclosure describes cannabis based medicinal extracts (CBMEs) and methods of treatment using the CBMEs to alleviate withdrawal effects associated with antidepressant medication cessation.

### CROSS-REFERENCE TO RELATED APPLICATIONS

This application is a continuation of U.S. application Ser. No. 15/436,480, filed Feb. 17, 2017, which claims benefit of priority from U.S. Provisional Application No. 62/296,410, filed Feb. 17, 2016. The disclosures of the prior applications are considered part of, and are incorporated by reference in, the disclosure of the present application.

### BACKGROUND

The present invention relates to the use of a combination of cannabinoids for the treatment and mitigation of symptoms associated with antidepressant dependence and/or withdrawal. Preferably the combination of cannabinoids is at least cannabidiol (CBD) and delta-9-tetrahydrocannabinol (THC).

Antidepressants, including selective serotonin reuptake inhibitors (SSRI) are a class of pharmaceuticals with alleged utility for a variety of psychiatric conditions including but not limited to depression, anxiety, obsessive compulsive disorder, and several others. Antidepressants were the third most common prescription drug taken by Americans of all ages in 2005-2008 and the most frequently used by persons aged 18-44 year. From 1988-1994 through 2005-2008, the rate of antidepressant uses in the United States among all ages increased nearly 400%. As of 2008 about 1 in 10 Americans aged 12 and over takes antidepressant medication, of which SSRIs are the most prevalent. About 14% of Americans taking antidepressant medication have done so for 10 years or longer.

Despite the enormity of antidepressant use in the United States and throughout the world, recent evidence has raised serious concerns about the efficacy of these drugs. As one example, a 2010 study in the Journal of the American Medical Association reported that SSRI antidepressants are not better than placebo for most cases of depression. In that study, the authors reviewed 30 years of data and concluded that “the benefit of antidepressant medication compared with placebo may be minimal or nonexistent in patients with mild or moderate symptoms”. Even where statistical significance of benefit has been found, there is substantial debate in the medical community regarding whether this statistical significance is associated with meaningful clinical significance.

While generally considered a safe medication, antidepressants are associated with a constellation of serious side effects. Some of the most common include sexual dysfunction, weight gain, and sleep disturbances. Another troubling issue with SSRIs is an increased risk of suicide which triggered the FDA to require its most serious warning, a “black box warning” on several SSRI medications. Another serious issue with SSRIs is the risk of persistent pulmonary hypertension of the newborn (PPHN) which has been reported in the offspring of some pregnant women taking SSRIs.

With substantial questions being raised about the efficacy of antidepressants, and the side effects associated with their use, many patients would like to discontinue their use. Unfortunately, discontinuing use of antidepressants can be extremely difficult. Interruption of treatment with an anti-depressant medication is associated with an antidepressant discontinuation syndrome; in early reports, it was referred to as a “withdrawal reaction.” Symptoms of antidepressant discontinuation syndrome can include flu-like symptoms, insomnia, nausea, imbalance, sensory disturbances, and hyperarousal. All approved anti-depressant agents have had case reports or warnings from their manufacturers of such reactions occurring in response to either abrupt discontinuation or medication tapering. These medications include selective serotonin reuptake inhibitors (SSRIs), tricyclic antidepressants, monoamine oxidase inhibitors (MAOIs), and atypical agents such as venlafaxine (Effexor), mirtazapine (Remeron), trazodone (Desyrel), and duloxetine (Cymbalta).

### SUMMARY

As specified in the Background Section above, there is a need in the art to develop new therapeutic tools to help patients mitigate the effects of antidepressant withdrawal and dependence.

Thus According to an aspect of the present invention there is provided the use of a combination of cannabinoids cannabidiol (CBD) and delta-9-tetrahydrocannabinol (THC) in the manufacture of a medicament for use in the treatment of antidepressant discontinuation syndrome (ADS), wherein the ratio of CBD:THC by weight is between 20:1 and 1:20.

The invention also provides a method of treating antidepressant discontinuation syndrome (ADS) in a human subject which comprises administering to a subject in need thereof a therapeutically effective amount of a medicament which contains a combination of cannabinoids cannabidiol (CBD) and delta-9-tetrahydrocannabinol (THC), wherein the ratio of CBD:THC by weight is between 20:1 and 1:20.

According to another aspect of the present invention there is provided the use of a combination of cannabinoids cannabidiol (CBD) and delta-9-tetrahydrocannabinol (THC) in the manufacture of a medicament for use in the treatment of physiological symptoms associated with the cessation of antidepressant use, wherein the ratio of CBD:THC by weight is between 20:1 and 1:20.

The invention also provides a method of treating the symptoms associated with the cessation of antidepressant use in a human subject which comprises administering to a subject in need thereof a therapeutically effective amount of a combination of the cannabinoids cannabidiol (CBD) and delta-9-tetrahydrocannabinol (THC), wherein the ratio of CBD:THC by weight is between 20:1 and 1:20.

According to another aspect of the present invention there is provided the use of a combination of cannabinoids cannabidiol (CBD) and delta-9-tetrahydrocannabinol (THC) in the manufacture of a medicament for use in the treatment of antidepressant dependence wherein the ratio of CBD:THC by weight is between 20:1 and 1:20.

The invention also provides a method of treating antidepressant dependence in a human subject which comprises administering to a subject in need thereof a therapeutically effective amount of a combination of cannabinoids cannabidiol (CBD) and delta-9-tetrahydrocannabinol (THC), wherein the ratio of CBD:THC by weight is between 20:1 and 1:20.

Preferred features of the invention will now be described in further detail. Features described as being preferred in relation to one aspect of the invention apply mutatis mutandis to all other aspects, unless clearly stated otherwise.

Preferably the use of the cannabinoids in the manufacture of a pharmaceutical formulation are for use in the treatment and/or prevention of antidepressant discontinuation syndrome.

Preferably the use of the cannabinoids in the manufacture of a pharmaceutical formulation are for use in the mitigation of symptoms associated with the cessation of antidepressant use.

Preferably the use of the cannabinoids in the manufacture of a pharmaceutical formulation are for use in the treatment of antidepressant dependence.

Preferably the ratio of CBD:THC by weight is between 20:1 and 1:20.

More preferably the ratio of CBD:THC by weight is between 10:1 and 1:10.

Favorably the cannabinoids are packaged for delivery in a titratable dosage form.

Preferably the cannabinoid CBD is administered separately, simultaneously, or sequentially to the cannabinoid THC.

The administration of a combination of cannabinoids such as THC and CBD to a patient could either be at the same time, wherein the cannabinoids would be contained in the same formulation. The cannabinoids could also be administered at separate times for example, a formulation containing CBD could be administered to a patient at a fixed time prior to a formulation containing THC in order to ameliorate some of the side effects of THC, which CBD is known to improve or vice versa. The two cannabinoids could also be administered consecutively to a patient if required.

The term “titrate” is defined as meaning that the patient is provided with a medication that is in such a form that smaller doses than the unit dose can be taken.

A “unit dose” is herein defined as a maximum dose of medication that can be taken at any one time or within a specified dosage period such as 3 hours. Titration of doses are beneficial to the patient as they are able to take smaller of doses of the medication until the drug is efficacious. It is understandable that not all patients will require exactly the same dose of medication, for example patients of a larger build or faster metabolism may require a higher dose than that required by a patient that is of a smaller build. Different patients may also present with different degrees of complaints and as such may require larger or smaller doses in order to treat the complaint effectively. The benefits of such a dosage form over dosage forms such as tablets, where smaller doses are difficult to take, are therefore evident.

Unit dose ranges are preferably in the range of between about 2 and 150 mg of each cannabinoid CBD and THC, more preferably in the range of about 5 to 50 mg of each cannabinoid and still more preferably from about 5 to 15 mg of each cannabinoid. In some embodiments it is understood that substantially higher doses of CBD are required.

Preferably the maximum daily dosage dose of medicament is less than or equal to 150 mg CBD and less than or equal to 130 mg THC. Preferably the cannabinoids are packaged for delivery such that delivery is targeted to an area selected from one or more of the following: oral, sublingual, buccal, oral, rectal, nasal and the pulmonary system.

More preferably the cannabinoids are in the form selected from one or more of the following: gel, gel spray, tablet, liquid, capsule, oil for vaporization and for nebulization, granulated whole plant matter or the like.

In order to accurately control the ratio of CBD and THC in the medicament a ratioed product is formulated from: a cannabis based medicinal extract which comprises THC at more than 90% of the total cannabinoid content in the extract; and a cannabis based medicinal extract which comprises CBD at more than 90% of the total cannabinoid content in the extract. These may then be mixed to the specified ratios.

Additionally, in some embodiments the pharmaceutical formulation further comprises one or more carrier solvents. Preferably the carrier solvents are medium chain triglycerides (MCT) ethanol and/or propylene glycol or other suitable carrier or excipient known to those having skill in the art.

In some embodiments the cannabinoids are present as a cannabis-based medicine extract (CBME). For example, the combination of cannabinoids comprises: a cannabis based medicinal extract which comprises THC at or about more than 70% of the total cannabinoid content in the extract; and a cannabis based medicinal extract which comprises CBD at or about more than 70% of the total cannabinoid content in the extract.

Optionally the combination of cannabinoids is substantially pure. Alternatively, the combination of cannabinoids is synthetic.

Preferably the THC and CBD are derived from plant extracts.

Cannabinoid-containing plant extracts will often comprise the major cannabinoid fraction and a minor cannabinoid fraction, along with several other cannabinoids. There will also be a non-cannabinoid fraction that will often contain components such as terpenes and other minor plant derived components including sterols, triglycerides, alkanes, squalene, tocopherol, and carotenoids.

For example a THC-containing plant extract may comprise between about 60 and 80% (w/w) THC in addition to CBD at 0.1-2.5% (w/w). The other cannabinoids include cannabigerol, cannabichromene, tetrahydrocannabidivarin, and tetrahydrocannabinolic acid. The non-cannabinoid fraction may comprise monoterpenes, di/tri-terpenes, sesquiterpenes, other terpenes and other minor plant components.

Furthermore a CBD-containing plant extract may comprise between 50 and 80% (w/w) CBD in addition to THC at .0.5-15% (w/w). The other cannabinoids include: cannabigerol cannabichromene, tetrahydrocannabidivarin, and tetrahydrocannabinolic acid. The non-cannabinoid fraction may comprise monoterpenes, di/tri-terpenes, sesquiterpenes, other terpenes and other minor plant components.

Furthermore a balanced THC-CBD plant extract may comprise between 25 and 40% (w/w) CBD in addition to THC at between 25 and 40% (w/w). The other cannabinoids include: cannabigerol cannabichromene, tetrahydrocannabidivarin, and tetrahydrocannabinolic acid. The non-cannabinoid fraction may comprise monoterpenes, di/tri-terpenes, sesquiterpenes, other terpenes and other minor plant components.

Preferably, the ratio of CBD and THC is between 100:1 and 1:100 and more preferably between 20:1 and 1:20 (CBD:THC) by weight.

In one embodiment the CBME are produced by extraction with supercritical or subcritical CO₂. In an alternative embodiment the CBME are produced by extraction from plant material by volatilization with a heated gas. In an alternative embodiment the CBME are produced from plant material through the sequential processes of CO₂ extraction followed by fractional distillation. In some embodiments, the CBME contain all of the naturally occurring cannabinoids, terpenes, and other minor plant components in the plant material (whole plant extract “WPE”). Alternatively synthetic or highly purified isolates of the cannabinoids can be used.

The term “approximately equal” is used to refer to ratios of cannabinoids which are in the range of between 0.9:1 to 1:0.9 (THC:CBD). Additionally, the term “1:1” is taken herein to refer to approximately equal amounts of cannabinoids:

According to another aspect of the present invention there is provided the use of a CBD type compound or derivative thereof and a THC type compound or derivative thereof in the manufacture of a pharmaceutical formulation for use in the treatment of the symptoms associated with antidepressant discontinuation, wherein the ratio of the CBD type compound or derivative thereof to THC type compound or derivative thereof by weight is between about 20:1 and 1:20.

Preferably the CBD type compound or derivative thereof is CBD, and the THC type compound or derivative thereof is THC.

According to another aspect of the present invention there is provided a method of treating the symptoms associated with antidepressant discontinuation in a human patient comprising administering to a patient in need thereof a therapeutically effective amount of one or more cannabinoids comprising cannabidiol (CBD) and delta-9-tetrahydrocannabinol (THC), wherein the ratio of CBD:THC by weight is between 20:1 and 1:20.

Preferably the antidepressant is an SSRI.

Alternatively the antidepressant is an SNRI or MAOI.

Preferably the CBD and THC are plant extracts.

### BRIEF DESCRIPTION OF THE DRAWINGS

FIG. 1 shows a summary of study participant responses to the question of whether they found cannabis helpful in reducing the withdrawal effects of antidepressant cessation.

FIG. 2 shows a summary of study participant responses to the question of whether cannabis made it easier to discontinue SSRI use.

FIG. 3 shows a summary of study participants responses to how cannabis alleviated specific symptoms associated with SSRI discontinuation.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context

### A. Preparation of Cannabis-Based Medicine Extracts (CBME)

Medicinal cannabis suitable for use in various embodiments of the invention described herein may be produced and prepared with reference to methods known by those having skill in the art. In one embodiment, harvested plant material can be processed as described in the procedure below. The process of manufacture of a High THC or High CBD cannabis-based medicine extract is described below.

### Medicinal Cannabis (High THC or High CBD)

Chopping to predominantly 2-3 mm;

Heating at 100-150° C. for sufficient time to decarboxylate the acid form of cannabinoids to produce neutral cannabinoids;

Extraction liquid carbon dioxide over 6 to 8 hours;

Removal of CO₂ by depressurization to recover crude extract;

Winterization followed by chilling (−20° C./48 h) to precipitate unwanted waxes;

Removal of unwanted waxy material by cold filtration; and

Removal of ethanol from the filtrate by thin film evaporation under reduced pressure

The resulting extract is referred to as a cannabis based medicinal drug extract and is also classified as a Botanical Drug Substance according to the US Food and Drug Administration Guidance for Industry Botanical Drug Products.

The quantity of cannabinoid in the CBME can be accurately assessed by way of measurement by HPLC with reference to the method disclosed in WO 02/064109 (Example 16), the contents of which are incorporated herein in their entirety by reference.

### EXAMPLE

### Study on the Efficacy of CBME and/or Cannabis on the Effects of Antidepressant Withdrawal

The study population was a group of 6 medical marijuana patients, all of whom had utilized medical marijuana during the withdrawal process from a selective serotonin reuptake inhibitor. The majority of patients had attempted SSRI cessation on more than one occasion without the concomitant use of medical cannabis. Medical marijuana patients that participated in the study provided their own medical marijuana.

Surprisingly treatment with medical cannabis was shown to produce a significant improvement in SSRI discontinuation syndrome symptoms.

### Results:

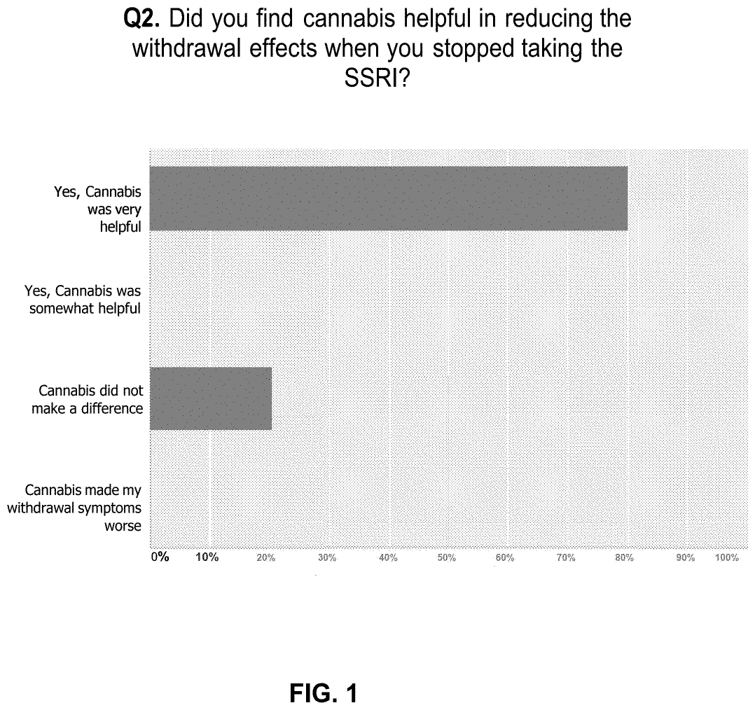

FIG. 1 shows the responses of the medical cannabis patients to the question of whether they found medical cannabis helpful in reducing the withdrawal effects of SSRI discontinuation. Approximately 80% of the patients surveyed reported that medical cannabis was “very helpful” in reducing the symptoms associated with SSRI discontinuation. Approximately 20% of participants reported that cannabis did not make a difference in reducing the symptoms associated with SSRI discontinuation. No participants reported that medical cannabis was “somewhat helpful” in reducing the symptoms associated with SSRI discontinuation and no participants reported that medical cannabis made their discontinuation symptoms worse.

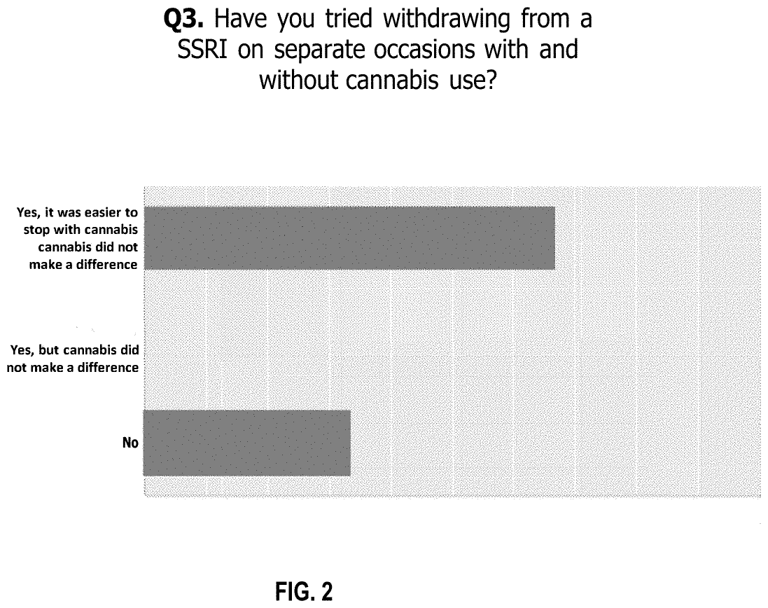

FIG. 2 shows the responses of the medical cannabis patients to the question of whether they had tried withdrawing from an SSRI on separate occasions with and without the concomitant use of medical cannabis. Approximately 70% of respondents reported that they had attempted to discontinue SSRI use on more than one occasion and that the use of medical cannabis made SSRI cessation easier.

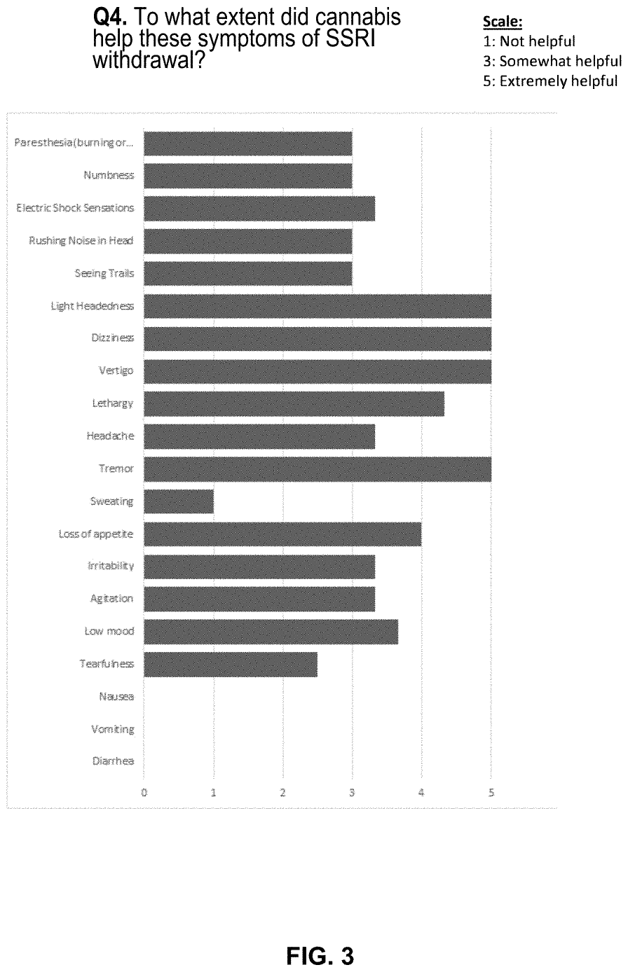

FIG. 3 shows the responses of the medical cannabis patients to the question of whether and to what extent medical cannabis helped alleviate specific symptoms of SSRI discontinuation. Generally, respondents reported that medical cannabis was helpful with alleviating paresthesia, numbness, electric shock sensations, rushing noise in the head, seeing trails, headache, loss of appetite, irritability, agitation, and tearfulness. Generally, respondents reported that medical cannabis was very helpful in alleviating light headedness, dizziness, vertigo, lethargy, tremor, loss of appetite and low mood.

It can therefore be concluded that a medication that contains THC and CBD offers a new treatment option in the treatment of patients with SSRI discontinuation syndrome and offers a new approach to help patients wishing to discontinue antidepressant use.

While the present disclosure includes many embodiments shown and described in detail, various modifications and improvements thereon will become readily apparent to those skilled in the art. Accordingly, the spirit and scope of the present invention is not to be limited by the foregoing examples but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

What is claimed is:

1. A method of mitigating an electric shock sensation associated with tapering or stopping antidepressant medication in a subject in need thereof, the method comprising:
administering an effective amount of a combination of cannabinoids including cannabidiol (CBD) and delta-9-tetrahydrocannabinol (THC), whereby the subject is less likely to experience the sensation of electric shocks than a corresponding subject tapering or stopping antidepressant medication without concomitant use of the combination, wherein the subject does not have symptoms of depression.

2. The method of claim 1, wherein the combination comprises a cannabis-based medicinal extract (CBME) comprising THC and CBD at a ratio of THC:CBD between 1:20 and 20:1 by weight, optionally wherein the combination is a ratioed product comprising a first CBME having more than 90% THC by weight based on total cannabinoid content; and a second CBME having more than 90% CBD by weight based on total cannabinoid content.

3. The method of claim 1, wherein the antidepressant medication is selected from the group consisting of a selective serotonin reuptake inhibitor (SSRI), a selective norepinephrine reuptake inhibitor (SNRI) and a monoamine oxidase inhibitor (MAOI).

4. The method of claim 3, wherein the antidepressant medication is the SSRI.

5. The method of claim 1, wherein the THC and CBD of the combination are packaged for delivery in a titratable dosage form, and wherein administering comprises sublingual; buccal; parenteral; oral; rectal, nasal; pulmonary delivery, or a combination thereof.

6. The method of claim 1, wherein the subject had been taking the antidepressant medication for at least 10 years, wherein the subject has a medical history of a failed attempt to discontinue taking the antidepressant medication, or a combination thereof.

7. The method of claim 6, wherein the subject has a medical history of at least two failed attempts to discontinue taking the antidepressant medication.

8. A method of alleviating light-headedness associated with tapering or stopping antidepressant medication in a subject in need thereof, the method comprising:
administering an effective amount of a combination of cannabinoids including cannabidiol (CBD) and delta-9-tetrahydrocannabinol (THC), whereby the subject is less likely to experience light-headedness than a corresponding subject tapering or stopping antidepressant medication without concomitant use of the combination, wherein the subject does not have symptoms of depression.

9. The method of claim 8, wherein the combination comprises a cannabis-based medicinal extract (CBME) comprising THC and CBD at a ratio of THC:CBD between 1:20 and 20:1 by weight, optionally wherein the combination is a ratioed product comprising a first CBME having more than 90% THC by weight based on total cannabinoid content; and a second CBME having more than 90% CBD by weight based on total cannabinoid content.

10. The method of claim 8, wherein the antidepressant medication is selected from the group consisting of a selective serotonin reuptake inhibitor (SSRI), a selective norepinephrine reuptake inhibitor (SNRI) and a monoamine oxidase inhibitor (MAOI).

11. The method of claim 8, wherein the THC and CBD of the combination are packaged for delivery in a titratable dosage form, and wherein administering comprises sublingual; buccal; parenteral; oral; rectal, nasal; pulmonary delivery, or a combination thereof.

12. The method of claim 8, wherein the subject had been taking the antidepressant medication for at least 10 years, wherein the subject has a medical history of a failed attempt to discontinue antidepressant, or a combination thereof.

13. The method of claim 12, wherein the subject has a medical history of at least two failed attempts to discontinue taking the antidepressant medication.

14. A method of mitigating a perception of visual trails associated with tapering or stopping antidepressant medication in a subject in need thereof, the method comprising:
administering an effective amount of a combination of cannabinoids including cannabidiol (CBD) and delta-9-tetrahydrocannabinol (THC), whereby the subject is less likely to experience visual trails than a corresponding subject tapering or stopping antidepressant medication without concomitant use of the combination, wherein the subject does not have symptoms of depression.

15. The method of claim 14, wherein the combination comprises a cannabis-based medicinal extract (CBME) comprising THC and CBD at a ratio of THC:CBD between 1:20 and 20:1 by weight, optionally wherein the combination is a ratioed product comprising a first CBME having more than 90% THC by weight based on total cannabinoid content; and a second CBME having more than 90% CBD by weight based on total cannabinoid content.

16. The method of claim 14, wherein the antidepressant medication is selected from the group consisting of a selective serotonin reuptake inhibitor (SSRI), a selective norepinephrine reuptake inhibitor (SNRI) and a monoamine oxidase inhibitor (MAOI).

17. The method of claim 16, wherein the antidepressant medication is the SSRI.

18. The method of claim 14, wherein the THC and CBD of the combination are packaged for delivery in a titratable dosage form, and wherein administering comprises sublingual; buccal; parenteral; oral; rectal, nasal; pulmonary delivery, or a combination thereof.

19. The method of claim 14, wherein the subject had been taking the antidepressant medication for at least 10 years, wherein the subject has a medical history of a failed attempt to discontinue antidepressant, or a combination thereof.

20. The method of claim 19, wherein the subject has a medical history of at least two failed attempts to discontinue taking the antidepressant medication.
